(19) United States
(12) Patent Application Publication
(10) Pub. No.: US 2010/0105103 A1
(43) Pub. Date: Apr. 29, 2010

(54) ALCOHOL TOLERANT ESCHERICHIA COLI AND METHODS OF PREPARATION THEREOF

(75) Inventors: Hsueh-Fen Juan, Taipei City (TW); Hirotada Mori, Taipei City (TW); Hsin-Yi Chang, Taipei City (TW); Hsuan-Cheng Huang, Taipei City (TW); Tsui-Chin Huang, Taipei City (TW); James C. Liao, Los Angeles, CA (US)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/473,040

(22) Filed: May 27, 2009

(30) Foreign Application Priority Data
Oct. 29, 2008 (TW) 097141589

(51) Int. Cl. C12Q 1/18; C12N 1/20

(52) U.S. Cl. 435/32; 435/252.8

(57) ABSTRACT

The present invention relates to E. coli mutants, which have enhanced alcohols tolerance and can be used in production of alcohols through fermentation. The present invention also provides a novel method to prepare the alcohol-tolerant E. coli strains.

### BACKGROUND OF THE INVENTION

1. Field of the Invention

The present invention relates to an alcohol tolerant microorganism and in particular to an alcohol tolerant E. coli and the method of its production.

2. The Prior Arts

A cheaper, more environmental protective alternative energy sources is in great demand in the globe warmth, petroleum- and energy-shortage era. Butanol is an important chemical materials and an industrial solvent. In 2005, 1-butanol extracted from corn has been demonstrated in USA to replace gas as a fuel in car running without modifying the car engine. New energy source will be found if economic improvement in butanol production is achieved.

Butanol can be transported without high pressure vessels as natural gas, and is a better fuel substitution than ethanol since it can be blended with gasoline in the ratio of 10%-100%. It can be stored and shipped through existing fuel pipelines. Biomass production of butanol can be made from corn, lignin such as corn straw, rice and wheat straw. Traditionally, butanol is produced by acetone, butanol and ethanol (ABE) fermentation technology through Clostridium acetobutylicum. However, the ABE process was gradually downfallen since 1950 because the fermentation process is quite complicated and difficult to control. Therefore butanol is presently manufactured from petrochemical process. The yield of butanol from glucose through ABE fermentation technology is quite low, with conversion rate of 15% in general, rarely exceeding 25%. The production of butanol was limited by severe product inhibition. Butanol at a concentration of 1% can significantly inhibit cell growth and the fermentation process. Consequently, butanol concentration in conventional ABE fermentations is usually lower than 1.3%

In summary, butanol produced from Clostridium acetobutylicum via conventional ABE fermentation has no economic value. Therefore, the need of establishing a butanol-tolerant microorganism, such as E. coli, exists, which can be applied in mass production of alternative energy from microorganism.

### SUMMARY OF THE INVENTION

In order to solve the abovementioned technology problem, an object of the present invention is to provide an alcohol-tolerant microorganism, and a production method of the microorganism, in particular to an alcohol tolerant E. coli and the method of its production.

To fulfill the abovementioned purpose, the invention provides an alcohol tolerant E. coli, which is selected from the group consisting of: BCRC 910400 (E. coli JH007), BCRC 910401 (E. coli JH016), and BCRC 910402 (E. coli JH017). The E. coli strains JH007, JH016 and JH017 were designated by the inventor respectively. Among them, the growth of JH007 could only be inhibited by 1-butanol at least 5% (v/v), isobutanol at least 3.5%, 1-propanol at least 6%, isopropanol at least 8%, or ethanol at least 8%. The growth of JH016 would only be inhibited by 1-butanol at least 2.5% (v/v), isobutanol at least 4%, 1-propanol at least 5%, isopropanol at least 5%, or ethanol at least 10%. And the growth of JH017 would only be inhibited at concentrations of 1-butanol at least 2% (v/v), isobutanol at least 3.5%, 1-propanol at least 5%, isopropanol at least 5%, or ethanol at least 12%.

Another objective of the present invention is to provide a method for the preparation of an alcohol tolerant E. coli, which comprises the following steps: screening host cells having at least 20% higher survival rates than wild type in the presence of 2% 1-butanol treatment from E. coli single-gene deletion mutants, known as Keio Collection; transforming a plasmid into the host cell, wherein the plasmid contains a gene of highly expressed 1-butanol tolerant protein; and isolating a transformant from transformed host cells with increased survival rate in the presence of 2% 1-butanol treatment, wherein the host cell is an ydhF⁻ mutant, or a potG⁻ mutant and the vector is an IPTG-inducible pCA24N plasmid. And the highly expressed 1-butanol tolerant protein is obtained from 2D electrophoresis analysis with 2% 1-butanol treated wild type followed by MS/MS analysis. The gene of highly expressed 1-butanol tolerant protein is selected from the group consisting of PhoH, MdoG, YdfG, Hmp, YqhD, and TolB.

Yet another object is to provide a method for enhancing 1-butanol tolerant of an E. coli, which comprises: modifying the E. coli to increase expression of a gene selected from the group consisting of phoH, mdoG, ydfG, hmp, yqhD, and tolB, wherein the E. coli is an ydhF⁻ mutant, or a potG⁻ mutant, and the 1-butanol concentration is from 2% to 5% (v/v).

A still further objective is to provide a 1-butanol as well as other alcohol, such as isobutanol, 1-propanol, isopropanol, or ethanol, tolerant E. coli. The example of JH007 showed that the growth of JH007 would only be inhibited by isobutanol at least 3.5%, 1-propanol at least 6%, isopropanol at least 8%, or ethanol at least 8%. The growth of JH016 would only be inhibited by isobutanol at least 4%, 1-propanol at least 5%, isopropanol at least 5%, or ethanol at least 10%. And the growth of JH017 would only be inhibited by isobutanol at least 3.5%, 1-propanol at least 5%, isopropanol at least 5%, or ethanol at least 12%.

The alcohol-tolerant E. coli in the present invention can tolerate 1-butanol at the concentration up to 5%, while the growth of wild type E. coli was inhibited by 1-butanol at concentration of 1% during conventional fermentation process. Therefore, the alcohol-tolerant E. coli in the present invention can be applied in mass production of alternative energy source in order to reach the goals of cost saving and yield increasing. Furthermore, the alcohol-tolerant E. coli in the present invention can tolerate isobutanol, 1-propanol, isopropanol, or ethanol as well, which will be beneficial in industrial application.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1 shows the growth curve of wild type E. coli BW25113 in the presence of various concentrations (%, v/v) of 1-butanol.

FIG. 2A shows relationship between cell concentration and 1-butanol concentration during late log phase (180 min).

FIG. 2B shows relationship between cell concentration and 1-butanol concentration during stationary phase (420 min).

FIG. 3 shows the survival rates of the thirteen mutant strains after 2% of 1-butanol treatment, wherein x-axis represents the type of mutant and Y-axis represents the survival rates in relative to the wild type.

FIG. 4 shows the survival rates of the thirteen mutant strains after 1% or 2% of 1-butanol treatment, wherein x-axis represents the type of mutant and Y-axis represents the survival rates in relative to the IPTG-uninduced strain.

FIG. 5 upper panel shows the DNA electrophoresis of plasmid extracted from the ASKA trasformants and the JH transformants.

FIG. 5 lower panel shows the Western blot using anti-His tag of protein electrophoresis from the JH transformants.

FIG. 6 shows the survival rates of the eighteen JH mutant strains after 2% of 1-butanol treatment, wherein x-axis represents the type of JH mutant and Y-axis represents the survival rates in relative to the IPTG-uninduced strain.

FIG. 7A shows the growth curve of E. coli JH007 of the present invention in the presence of various concentration of 1-butanol.

FIGS. 7B, 7C, 7D and 7E show the growth curve of E. coli JH007 of the present invention in the presence of various concentration of alcohol.

FIGS. 8A, 8B, 8C, 8D and 8E show the growth curve of E. coli JH016 of the present invention in the presence of various concentrations of alcohols.

FIGS. 9A, 9B, 9C, 9D and 9E show the growth curve of E. coli JH017 of the present invention in the presence of various concentrations of alcohols.

FIG. 10A shows the morphology of wild type E. coli BW25113 under transmission electron microscopy (TEM).

FIG. 10B shows the morphology of E. coli JH007 under transmission electron microscopy (TEM).

### DETAILED DESCRIPTION OF THE PREFERRED EMBODIMENT

Systems biology is a new biological field, which aims to explain how higher level properties of complex biological systems arise using tools from mathematical logic and computer models in systematic science. This new field integrates many disciplines including biology, computer science, applied mathematics, physics and engineering to predict the performance of cell, organ, system, or even to the whole organism.

Recently, a set of single-gene knockout mutants of all the non-essential genes in E. coli K-12 (the Keio collection) was constructed. It has been applied in various physiological properties. The E. coli K-12 strain BW25113 was used in the present invention as a wild-type strain for the 1-butanol tolerance screening. The mutants of a strain derivative of W3110 (Baba et al., Molecular systems biology 2, 2006 0008, 2006) were obtained from a Keio collection of all the non-essential genes knockout mutants of BW25113. As a result, the present invention proposed that the deletion of the cell surface transporters was required for tolerate to 1-butanol in this strain. At the same time, several strains of ASKA library which contains each Escherichia coli open reading frame (ORF) were cloned into the expression vector pCA24N in order to develop the 1-butanol tolerance.

The alcohol tolerant E. coli JH007 (BCRC910400), E. coli JH016 (BCRC910401), and E. coli JH017 (BCRC910402) were obtained from screening of the systematic collection of single gene-disrupted E. coli K-12 mutants, the Keio collection. The 1-butanol-tolerant phenotype was obtained from screening of all the non-essential genes knockout mutants, followed by identification of 1-butanol tolerant related proteins using proteomics technology and understanding of the physiological responses of cells after the 1-butanol stimulus. Twenty two differentially expressed proteins were identified in the present invention. Among them, thirteen highly expressed genes were subcloned into IPTG-inducible vectors to determine the 1-butanol tolerance of the transformants. Six of them revealed higher 1-butanol tolerance than the normal control group among these highly expressed strains. Plasmids containing 1-butanol tolerance genes were transformed into the gene knockout mutants. Three E. coli strains JH007, JH016 and JH017 were shown to have superior 1-butanol tolerance. The strain JH007 demonstrated a 5.5 fold 1-butanol tolerance (2% v/v) than that of control, and the tolerance of 1-butanol concentration could be up to 5% (v/v). The strain JH016 demonstrated a best ethanol tolerance and the tolerance of ethanol concentration could be up to 12% (v/v). Therefore, these three E. coli strains JH007, JH016 and JH017 in the present invention can be applied in mass production of 1-butanol to generate biofuels. The details of establishing the alcohol tolerant E. coli and its analysis of morphology and characteristics are described as follows:

### MATERIALS AND METHODS

The alcohol tolerant E. coli JH007, E. coli JH016, and E. coli JH017 were deposited in Bioresource Collection and Research Center (BCRC, Hsin-Chu, Taiwan) with accession numbers of 910400, 910401 and 910402 respectively.

E. coli K-12 strain BW25113 (Δ(araD-araB)567, ΔlacZ4787(::rrnB-3), lambda⁻, rph-1, Δ(rhaD-rhaB)568, hsdR514) and isogenic deletion mutants of the Keio Collection were used. Plasmids pCA24N carrying htpG, ahpF, gpmA, wrbA, phoH, mdoG, ydfG, sodB, hchA, hmp, yqhD, grpE or tolB were obtained from ASKA library.

E. coli cells were routinely grown in LB media containing 1% Bacto Tryptone, 0.5% yeast extract, and 1% NaCl at 37° C. Antibiotics kanamycin or chloramphenicol was added into LB media when required at the final concentration of 30 μg/ml or 50 μg/ml, respectively. 1-butanol was added to the cells when the cell density reached 0.4 OD₆₀₀. Protein was extracted after 2 h of 1-butanol treatment.

### Example 1

### 1-Butanol Tolerance of Wild Type E. Coli Strain

E. coli wild-type strain BW25113 was cultured overnight and then inoculated into a fresh LB medium containing 0-10% (v/v) of 1-butanol. Cells were cultivated with shaking and the optical density at 600 nm was measured every 15 minutes.

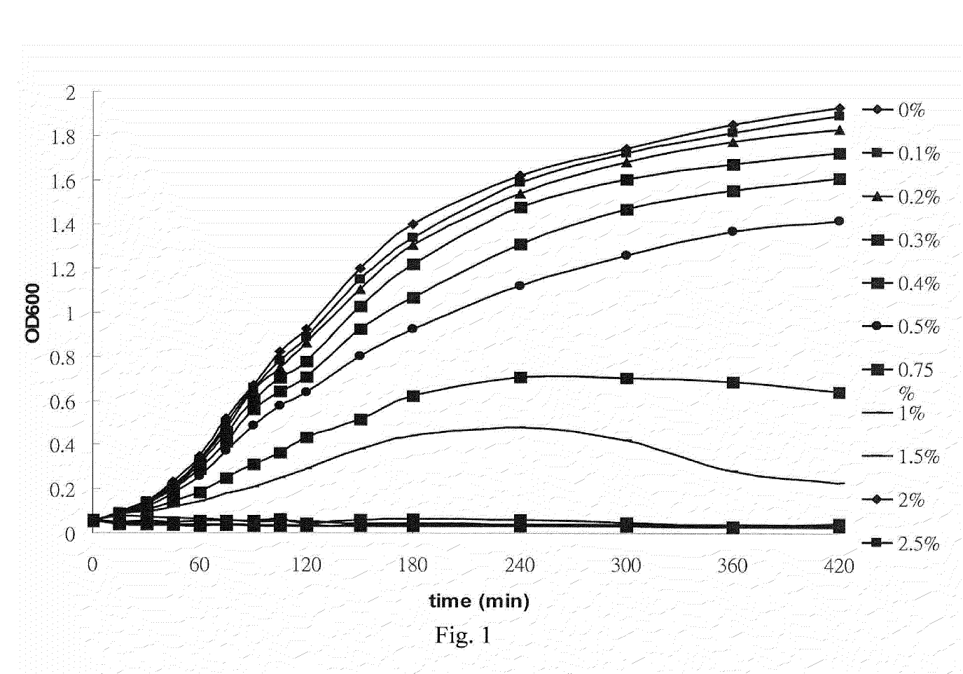

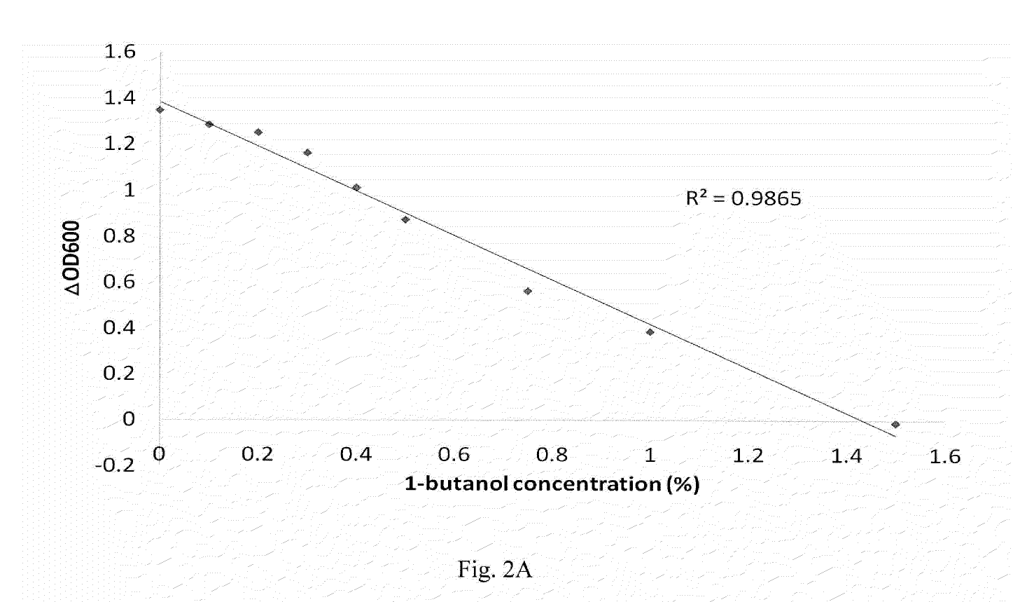

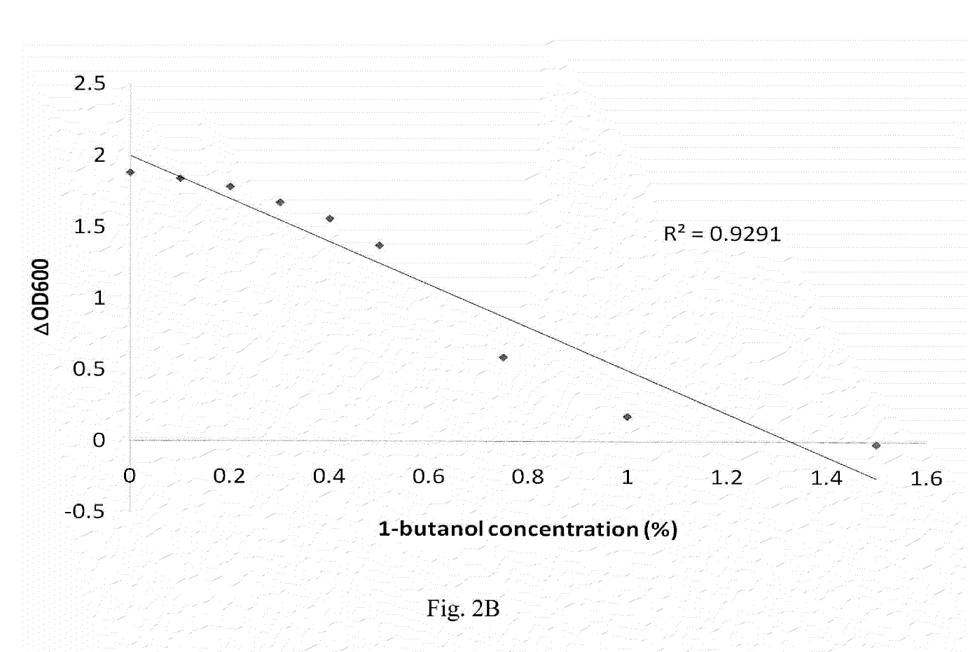

As shown in FIG. 1, the growth of E. coli was monitored by the optical density at 600 nm, which was based on the changes of cell turbidity in the present invention. The OD₆₀₀ decreased with the increasing concentration of 1-butanol. 1-butanol was shown to inhibit the cell density of E. coli. The growth inhibitory was even obvious when the concentration of 1-butanol was equal or larger than 1.5% (v/v). FIG. 2A and FIG. 2B showed the reverse linear effects of various concentration of 1-butanol toward the OD₆₀₀ at late log phase (180 min) or stationary phase (420 min) respectively, where the Pearson's correlation coefficient R² were 0.9865 and 0.9291 respectively. Therefore, the linear inhibitory effects of 1-butanol on E. coli BW25113 growth were shown in both late log phase and stationary phase, and the growth of wild type E. coli BW25113 was almost completely inhibited when the concentration of 1-butanol was equal or larger than 1.5% (v/v).

### Example 2

### Large Scale Screening of 3985 Single-Gene Deletion Mutants (the Keio Collection)

Mutants from the Keio collection were replicated into 96-well plates containing 100 μl of LB medium supplemented with 30 μg/ml of kanamycin per well. Plates were incubated overnight at 37° C. with shaking. A 10 μl of cell solution from overnight culture was inoculated into 96-well polystyrene plates containing 100 μl of LB medium as the control group or LB medium containing 2% 1-butanol as the experimental group at 37° C. for 3 h. Growth of cells was monitored by reading the absorbance (OD₅₉₅) of each well using a microplate reader (Bio-Rad, Hercules, Calif., USA). Then, the survival rate was obtained by dividing the OD₅₉₅ difference of the mutant strain (after 3 h of treatment) to that of the wild type strain.

Survival rate=difference of OD₅₉₅ between mutant (3 h-0 h)/difference of OD₅₉₅ between wild type (3 h-0 h)×100%

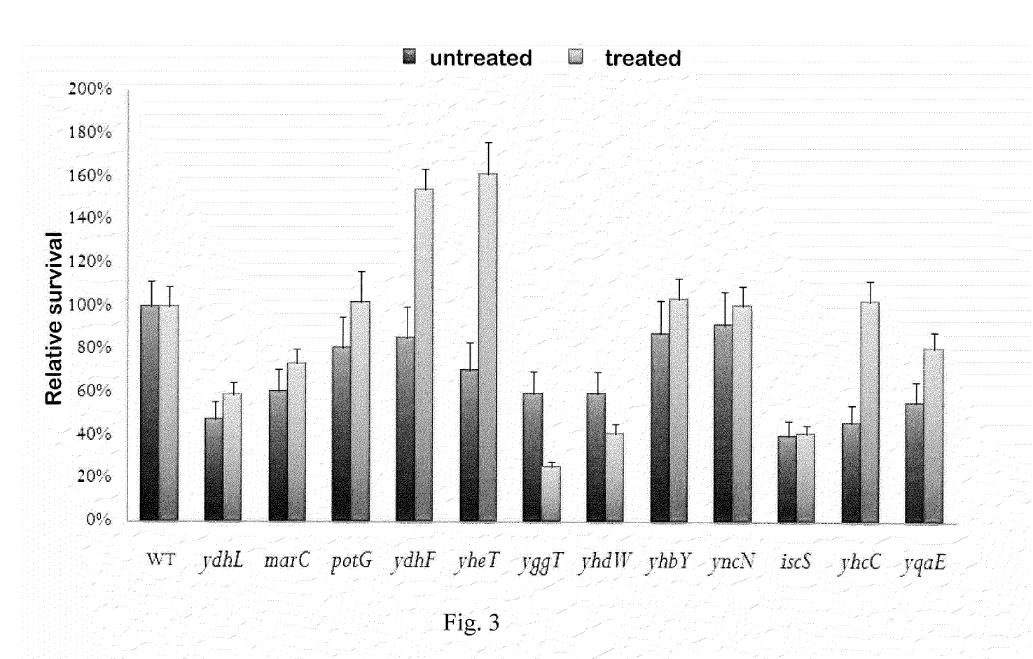

Mutants with higher survival rate than that of the wild type were selected as the candidate mutants. From 3985 mutants, 85 mutants were shown to be more tolerant to 1-butanol than the wild type. Among them, thirteen mutants had a survival rate higher than 20%. Three mutants, ydhF, potG⁻ and yheT⁻ showed the highest 1-butanol tolerance (FIG. 3, wherein x-axis represents the type of mutant and Y-axis represents the relative survival rates), which were used as host cells for the following experiment.

### Example 3

### Protein Extraction of 1-Butanol Tolerant Mutant Strains

Both the experimental group (after 2 h 1-butanol treatment) and the control group (untreated) of E. coli BW25113 from 20 ml of overnight culture to 0.4 OD₅₉₅ were collected and washed three times with a solution containing 3 mM of KCl, 1.5 mM of KH₂PO₄, 68 mM of NaCl, and 9 mM of NaH₂PO₄. One ml of lysis solution containing 7 M urea, 2 M thiourea, 4% CHAPS and 0.002% bromophenol blue was added to the cell pellet. The mixture was sonicated in discontinuous mode for 5 minutes on ice. The cell lysate was centrifuged at 4° C. at 15,000 g for 30 min. The supernatant was collected and the concentration was measured with a protein assay kit (Bio-Rad, Hercules, Calif., USA).

### Two Dimensional Gel Electrophoresis (2DE) of Protein

2DE was performed using an Ettan IPGphor III (GE). Four hundred μg of total proteins were mixed with rehydration buffer containing 7 M urea, 2 M thiourea, 4% CHAPS, 65 mM DTE, 1% pH 3-10 NL IPG buffer and 0.002% bromophenol blue to a total volume of 315 μl. The mixtures were loaded onto an 18 cm pH 4-7 NL gradient Immobiline DryStrip gels (Bio-Rad, Hercules, Calif., USA). IEF parameters for separation were 50 μA per strip at 20° C. with a rehydration step for 12 h. IEF was carried out under the following conditions: (1) 100 V for 1 h; (2) 250 V for 1 h; (3) 500 V for 1 h; (4) 1,000 V for 1 h; (5) 4,000 V for 1 h; and (6) 8,000 V for 65 kVh. After reduction with 65 mM DTE and alkylation with 55 mM iodoacetamide, the second-dimensional separation was performed on a 12.5% homogeneous polyacrylamide gel. The protein gels were fixed in 10% methanol/7% acetic acid and stained using the SYPRO® Ruby method (molecular probe). Gels were then scanned using a Typhoon 9400™ Fluorescence Imager (GE) and analyzed by Image Master™ 2D elite software package (GE) using high image quality TIF format.

The 2D image of the non-treated cells was set as the reference image. The protein spots which were detected only on the 1-butanol treated cells after matching the gel image with the reference image were excised and washed with a solution containing 50 mM of ammonium bicarbonate and ACN at a 1:1 ratio (v/v). After treatment with Na₂CO₃, protein spots were digested at 37° C. with trypsin for 16 h. The resulting peptides were extracted from the gel with 1% TFA in 50% ACN. The extracts were combined and evaporated to dryness, dissolved in 2% ACN containing 0.1% TFA and directly loaded onto the sample plate of a MALDI-TOF mass spectrometer. MALDI-TOF MS or MS/MS were performed on a dedicated Q-TOF Ultima MALDI instrument (Micromass, Manchester, UK) for molecular weight determination. Subsequently, proteins were identified in the SWISS-PROT database.

Twenty-two differentially expressed proteins were identified after 2DE and MALDI-TOF MS analysis in the present invention. Highly expressed proteins include the anti-oxidative enzymes, chaperones, and membrane transporters. On the other hand, proteins involved in glycolysis, arginine degradation, tryptophan degradation, ATP synthesis, ATP transportation and membrane signaling transduction were down-regulated.

### Example 4

### Proteins with High 1-Butanol Tolerance

Thirteen of the abovementioned highly expressed protein genes identified from the 2DE experiment were subcloned into IPTG-inducible plasmids respectively. The abovementioned plasmids were transfected into cells and cultivated overnight with LB media containing chloraphenicol. IPTG were added into the media at the final concentration of 0.1 mM. The cultures were diluted in a fresh LB medium supplemented with 50 μg/ml chloramphenicol, with or without the addition of 1% or 2% of 1-butanol in triplicate. Cells were incubated at 37° C. with shaking and the optical density was measured at the beginning and after 4 h. The survival rates were calculated using the following formula.

Survival rate=difference of OD₅₉₅ between mutants with IPTG induced (4 h-0 h)/difference of OD₅₉₅ between mutants without IPTG induced (4 h-0 h)×100%

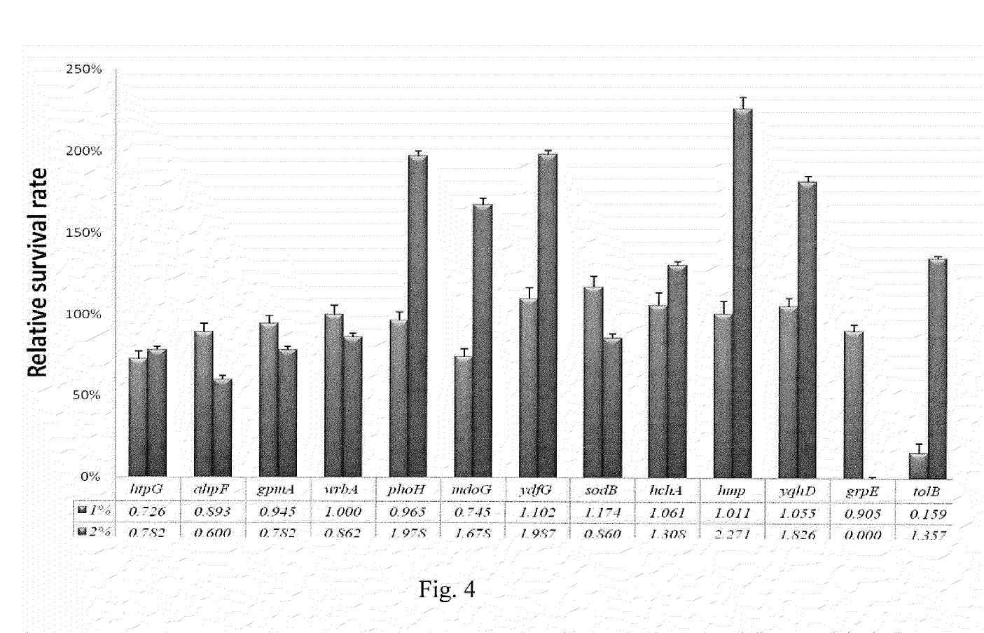

Referring to FIG. 4, wherein x-axis represents the type of mutant and Y-axis represents the survival rates of the 1% 1-butanol treated strain or 2% 2-butanol treated strain in relative to the uninduced strain. Six of the highly expressed strains showed higher 1-butanol tolerance, which was related to the function of membrane synthesis or anti-oxidation. Therefore, the 1-butanol tolerance was enhanced by strengthening the defense capability of membrane or keeping the cells away from harm.

### Example 5

### Protein Expression and the Butanol Tolerance of JH Strains

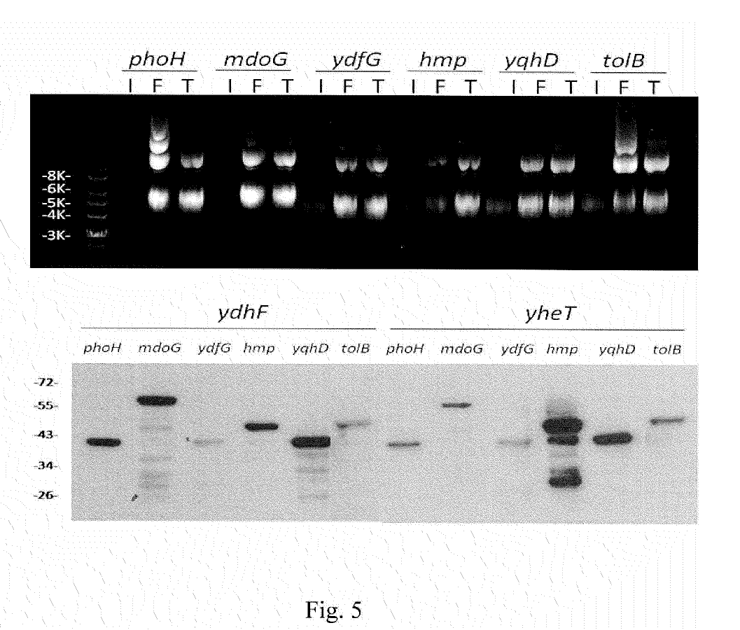

The present invention screened out the 1-butanol tolerant proteins, and transformed genes of these proteins into Keio mutants selected from the screening experiment respectively to yield transformants. The plasmids carrying phoH, mdoG, ydfG, hmp, yqhD, and tolB gene were sent into the ydhF⁻, potG⁻, and yheT⁻ Keio mutants by electrotransformation (upper panel of FIG. 5, wherein I indicated plasmid from ASKA; F indicated plasmid from ydhF⁻ mutant and T indicated plasmid from yheT⁻ mutant). Overnight cultures of E. coli of both mutants grown at 37° C. in YT10 broth supplemented with 30 μg/ml of kanamycin were diluted 1:10 in 500 ml of YT10 supplemented with 30 μg/ml of kanamycin. Cells were harvested by centrifugation at 4,000×g for 10 min when the optical density at 600 nm was between 0.4 and 0.6. The cells were washed with ice-cold distilled water three times sequentially by centrifugation at 5,000×g, 6,000×g, and 7,000×g. After the final wash of 10% glycerol followed by 8,000×g centrifugation, the cells were suspended in 10% glycerol and either stored at −80° C. or immediately used for electroporation at a concentration of 10¹⁰ cells/ml using a MicroPulser Electroporator (Bio-Rad, Hercules, Calif., USA). Electroporation was performed by a single pulse at 1.7 kV, 200Ω, and 25 μF. The electroporated cell suspension was diluted with 0.1 ml of a LB medium and incubated at 37° C. for 30 min before being placed on a LB agar supplemented with 30 μg/ml of kanamycin and 50 μg/ml of chloramphenicol.

The protein expressions were then confirmed by western blotting analysis. Briefly, the proteins were extracted with lysis buffer and the lysates of the cells were subjected to electrophoresis, transferred to polyvinyl-difluoride membranes. After blocking for 1 h with gentle shaking, membranes were probed with primary antibodies of His-tag and secondary antibodies of anti-mouse IgG (FIG. 5, lower panel).

### Identification of Butanol-Tolerant JH Transformants

JH transformants were highly butanol-tolerant strains obtained from large-scale screening and plasmid transformation. The JH strains used in this invention were listed in Table 1.

TABLE 1

| | | Highly expressed |
|---|---|---|
| JH transformant | Knock-out gene | protein |
| JH001 | yheT⁻ | phoH |
| JH002 | yheT⁻ | mdoG |
| JH003 | yheT⁻ | ydfG |
| JH004 | yheT⁻ | hmp |
| JH005 | yheT⁻ | yqhD |
| JH006 | yheT⁻ | tolB |
| JH007 | ydhF⁻ | phoH |
| JH008 | ydhF⁻ | mdoG |
| JH009 | ydhF⁻ | ydfG |
| JH010 | ydhF⁻ | hmp |
| JH011 | ydhF⁻ | yqhD |
| JH012 | ydhF⁻ | tolB |
| JH013 | potG⁻ | phoH |
| JH014 | potG⁻ | mdoG |
| JH015 | potG⁻ | ydfG |
| JH016 | potG⁻ | hmp |
| JH017 | potG⁻ | yqhD |
| JH018 | potG⁻ | tolB |

These 18 strains were cultivated overnight then inoculated into LB media supplemented with 30 μg/ml of kanamycin and 50 μg/ml of chloraphenicol. IPTG were added into the media at the final concentration of 0.1 mM. After 2 h of induction, the cultures were diluted in a fresh LB medium supplemented with chloramphenicol, with or without the addition of 2% of 1-butanol in triplicate. Cells were incubated at 37° C. with shaking and the optical density was measured at the beginning and after 4 h. The survival rates were calculated using the following formula.

Survival rate=difference of OD₅₉₅ between mutants with IPTG induced (4 h-0 h)/difference of OD₅₉₅ between mutants without IPTG induced (4 h-0 h)×100%

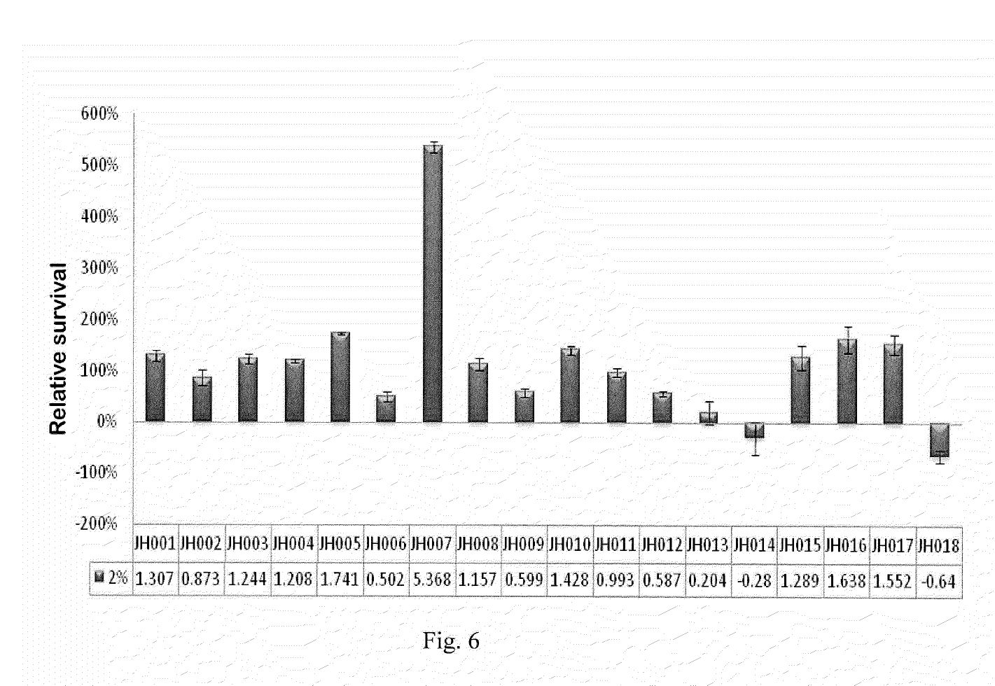

Referring to FIG. 6, wherein x-axis is the type of mutant and Y-axis is the survival rates of the butanol treated strain in relative to the uninduced strain. Most of the JH strains produced highly expressed proteins such as PhoH, MdoG, YdfG, Hmp, YqhD, and TolB. These strains showed better 1-butanol tolerance than the control group. Among them, the strain JH007 demonstrated a 5.5 fold 1-butanol tolerance (2% v/v) than that of control, which is an ydhF⁻ mutant and overexpressing PhoH. The strain JH016 (a potG⁻ mutant and overexpressing Hmp) and the strain JH017 (a potG⁻ mutant and overexpressing YqhD) also showed a high 1-butanol tolerance.

### Example 6

### The Growth Curves of JH Strains Showed Alcohol Tolerance

Single colony of the JH007 strain was cultivated overnight then inoculated into LB media supplemented with 30 μg/ml of kanamycin and 50 μg/ml of chloraphenicol. IPTG were added into the media at the final concentration of 0.1 mM when OD reached 0.6. After 2 h of induction, the cultures were diluted to 0.35 OD₅₉₅ in a fresh LB medium supplemented with kanamycin and 50 μg/ml of chloraphenicol, with the addition of 0-18% ethanol, 0-5% 1-propanol, 0-5% iso-propanol, 0-7% 1-butanol, and 0-7% iso-butanol in triplicate. Cells were incubated at 37° C. with shaking and the optical density was determined every 30 min and plotted.

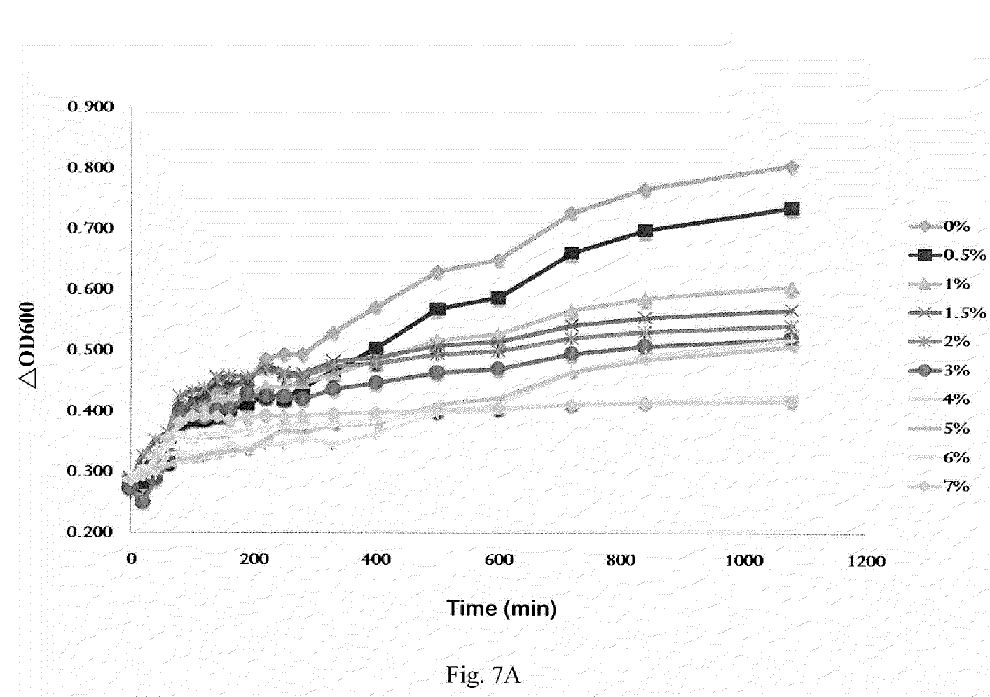

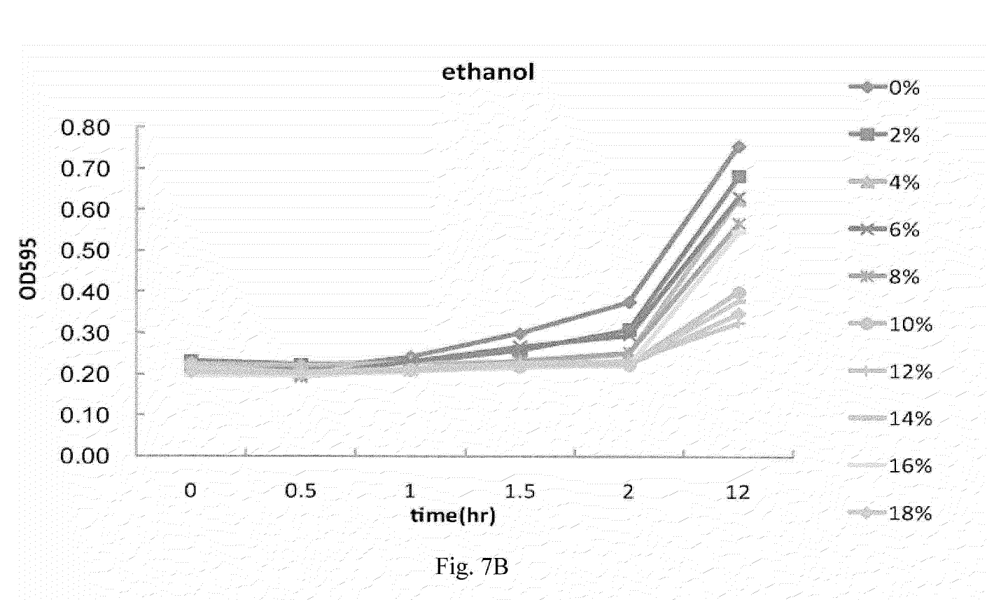

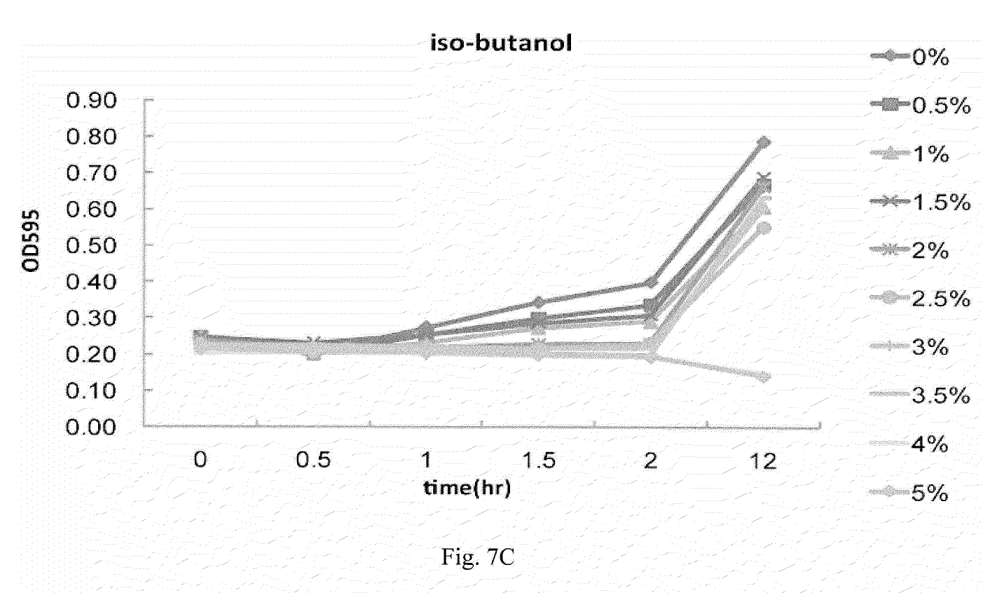

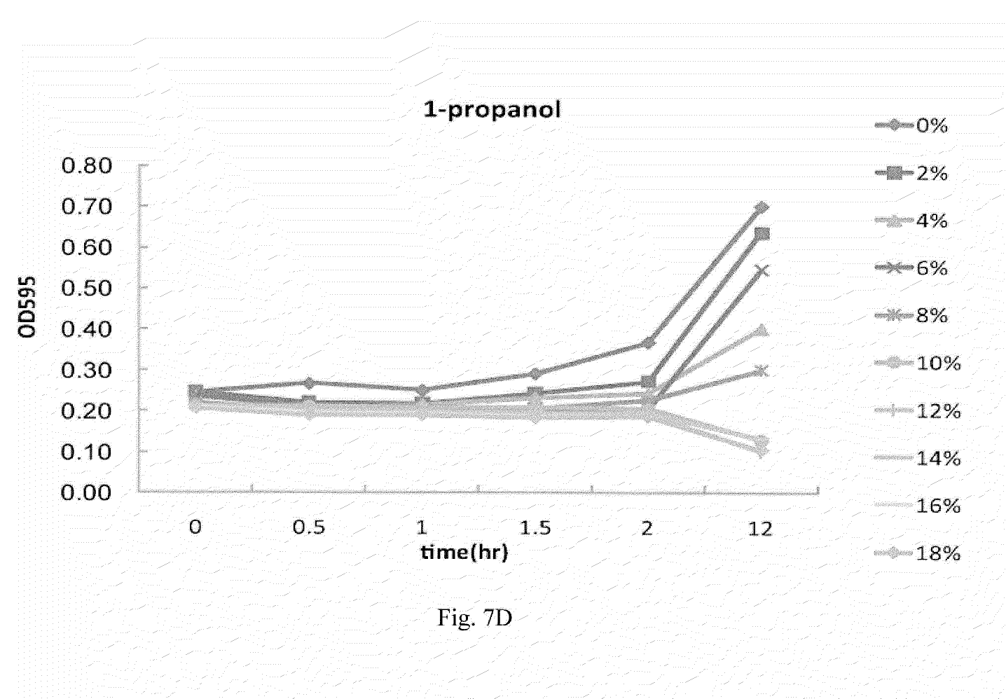

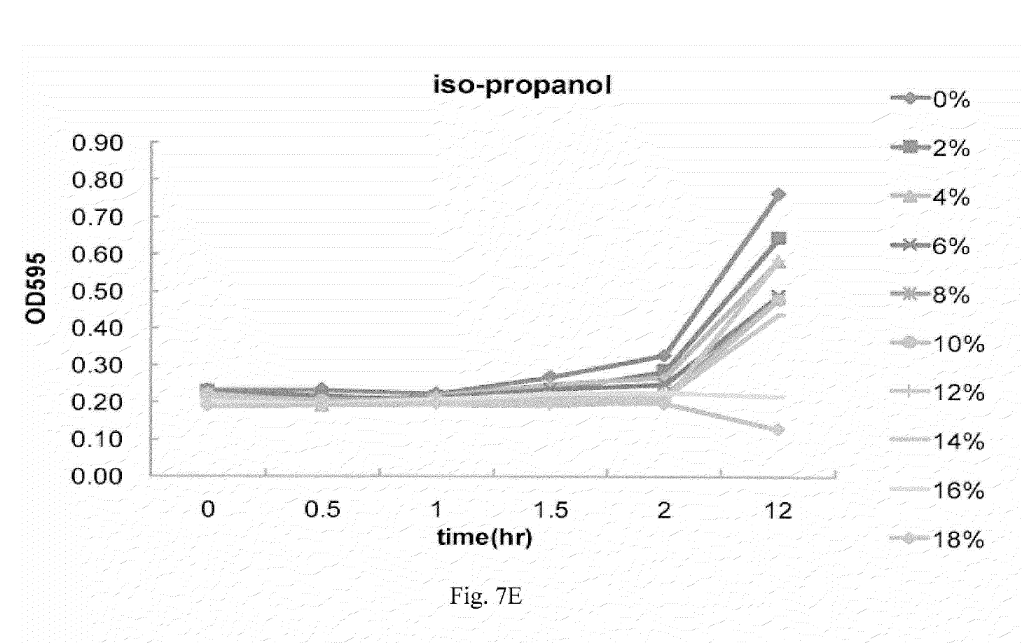

Referring to FIG. 7, cell growth condition of JH007 strain was monitored with OD₅₉₅ with the changes of cell turbidities. The OD₅₉₅ values showed concentration-dependent decrease with the increase of alcohol concentration. It appeared that the growth density of JH007 strain was partially inhibited by alcohol. The growth of JH007 was inhibited by 1-butanol at concentrations at least 5% (v/v), isobutanol at least 3.5%, 1-propanol at least 6%, isopropanol at least 8%, or ethanol at least 8%, which is superior to the wild type strain.

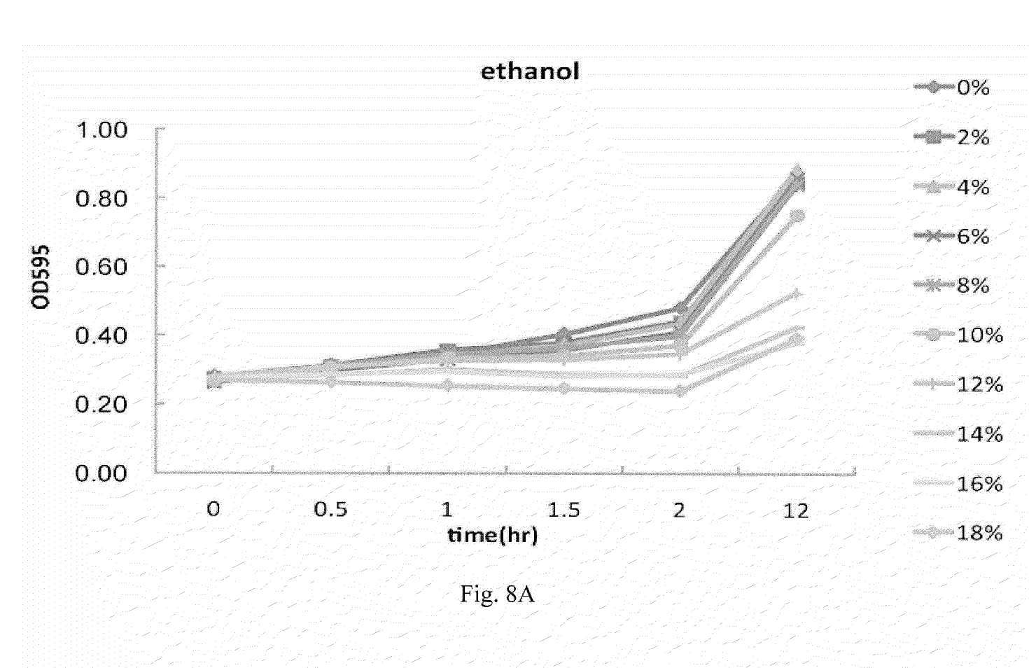

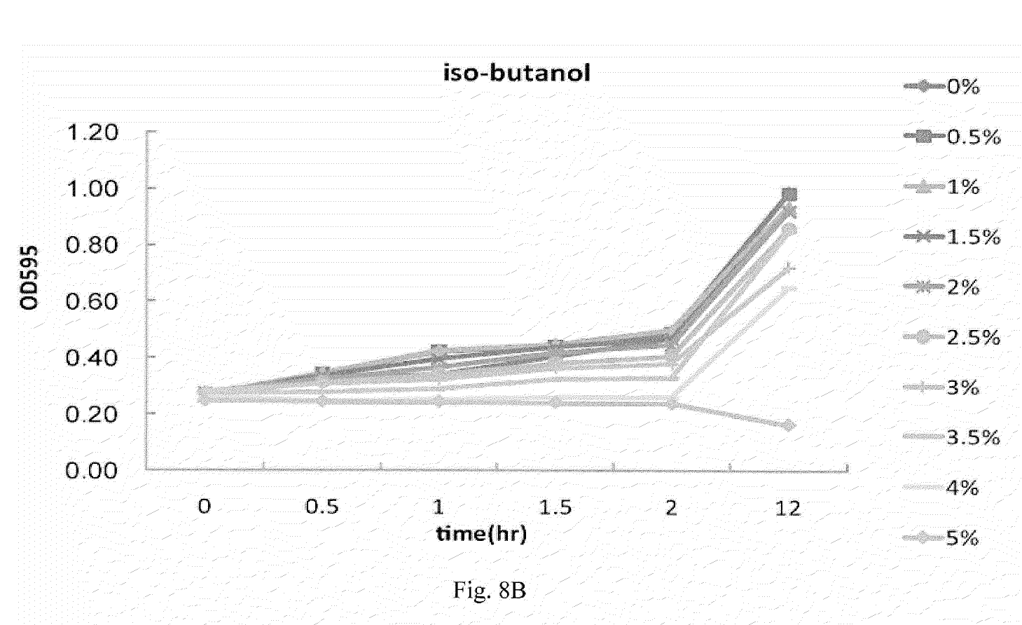

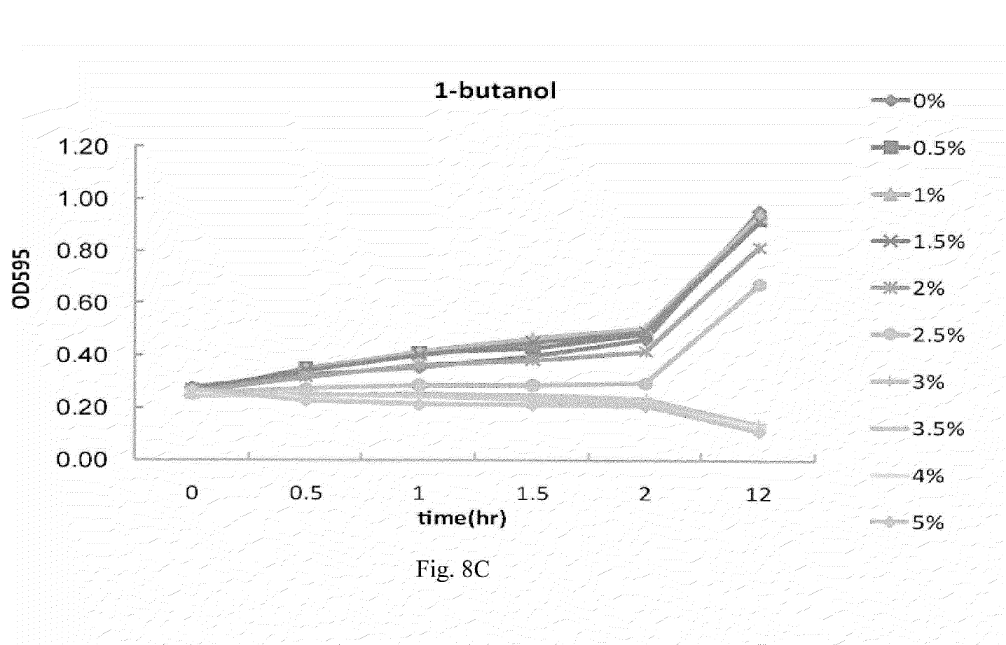

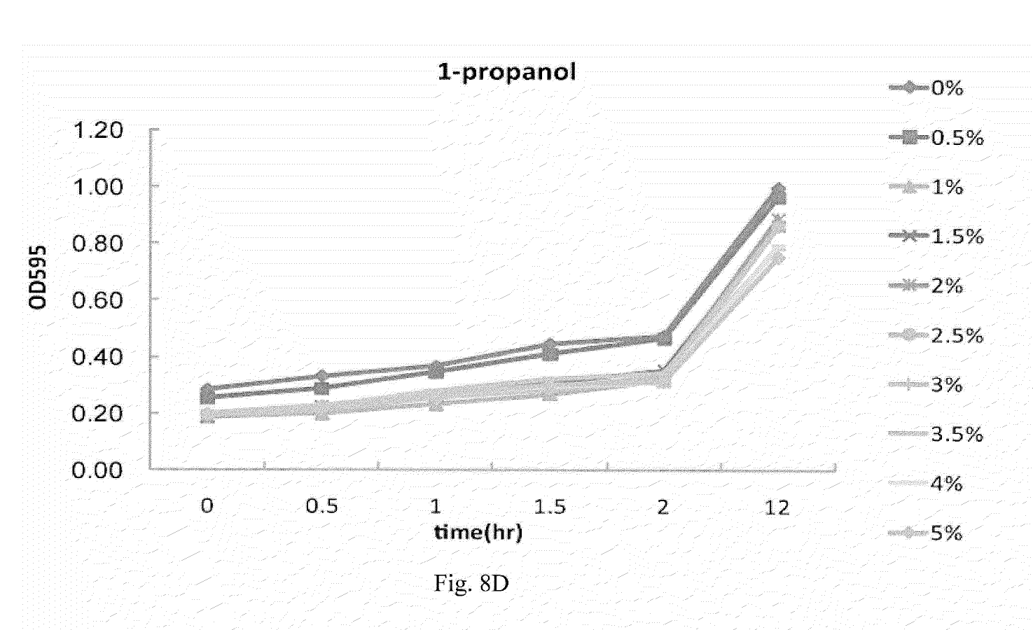

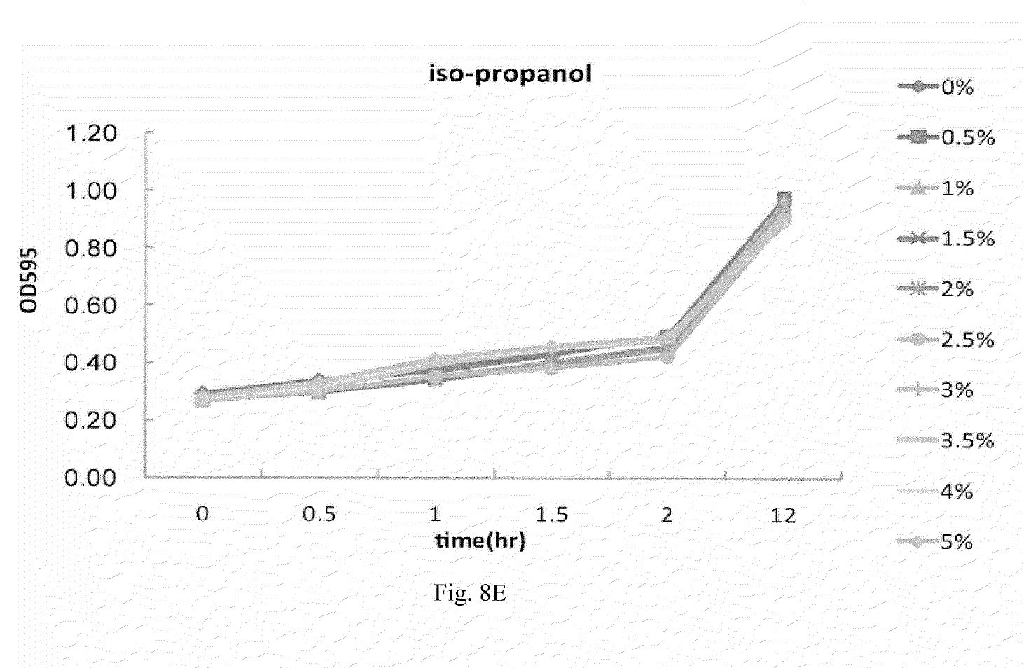

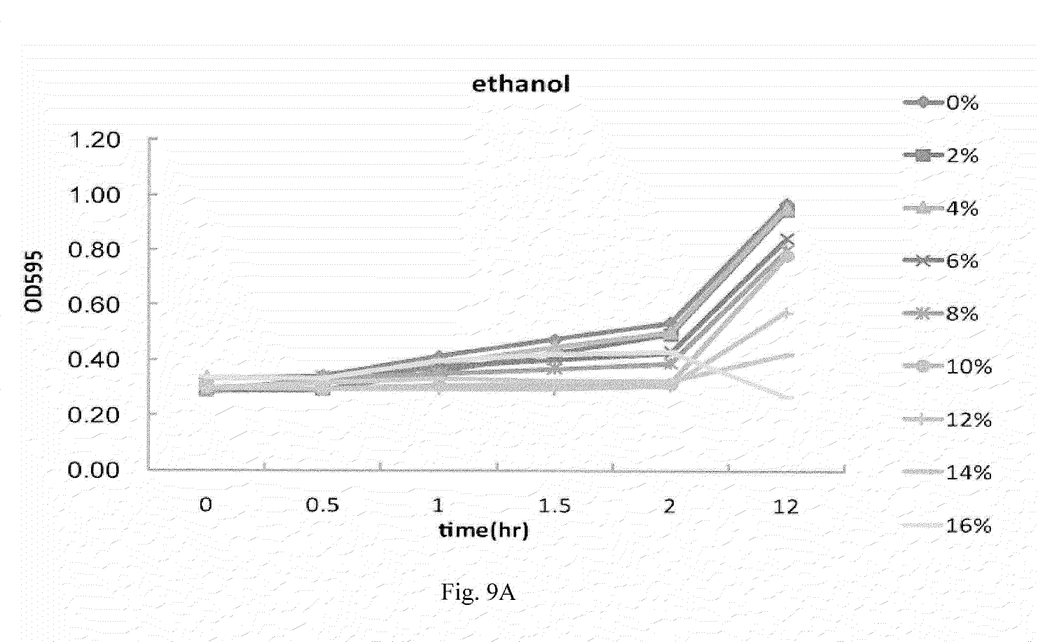

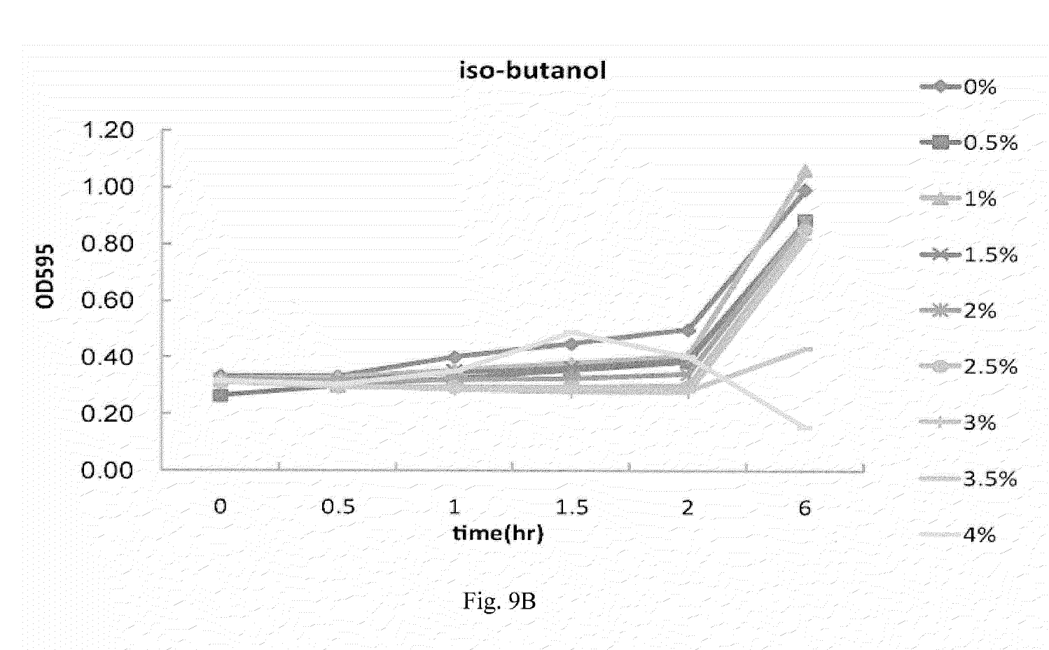

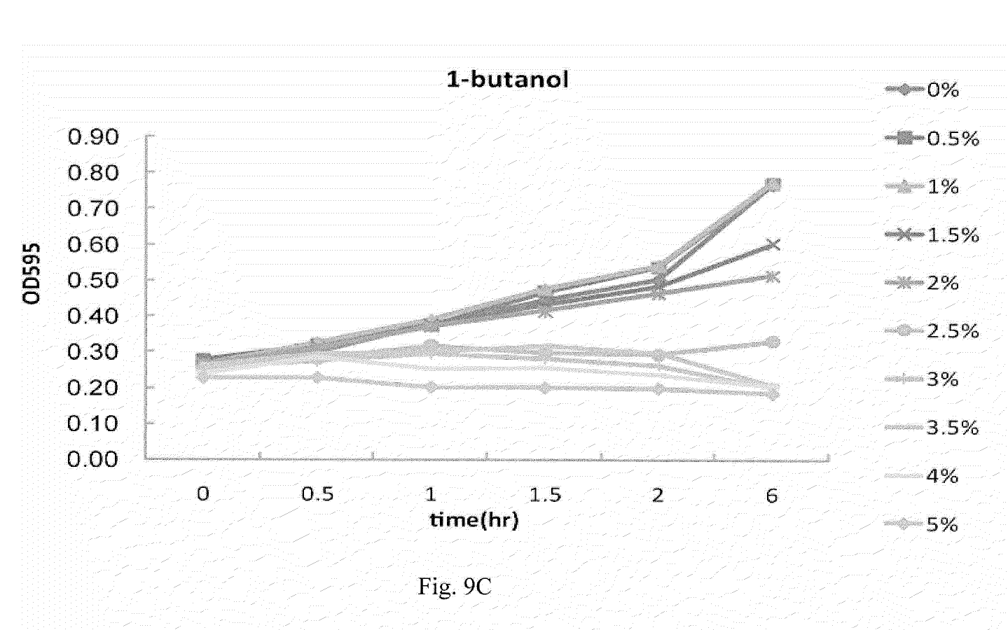

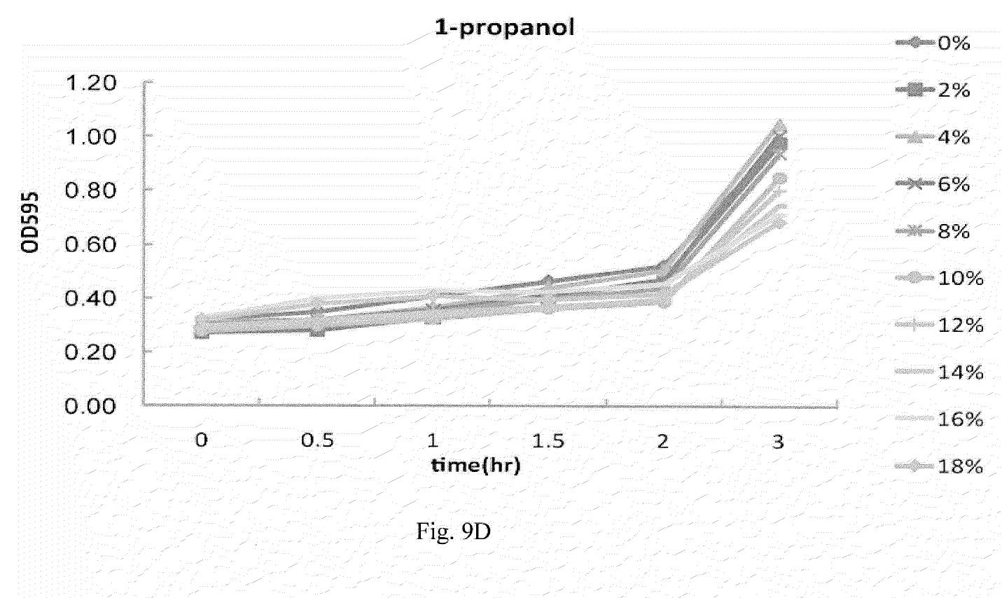

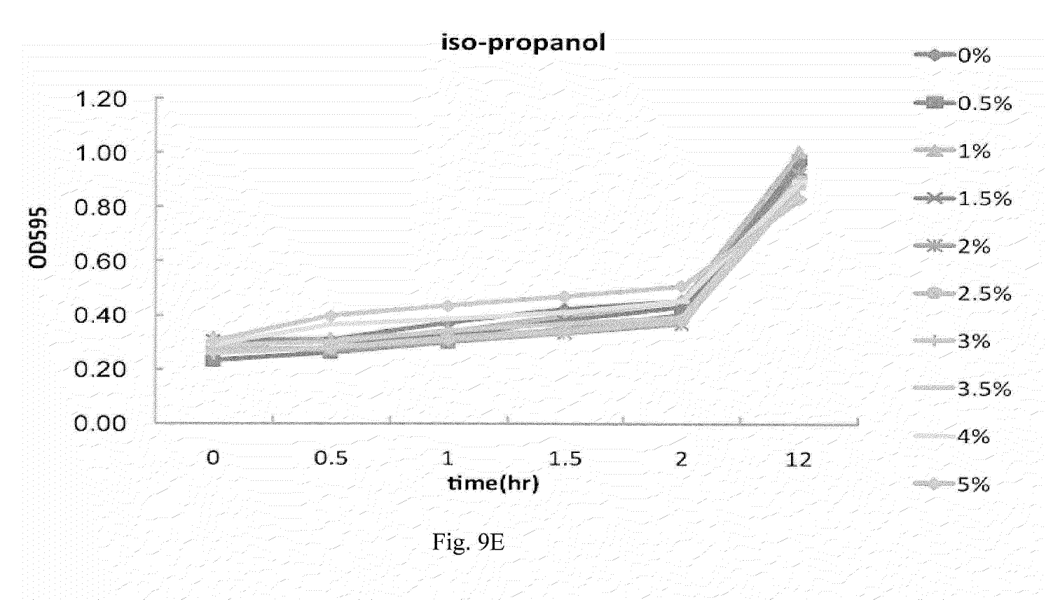

FIG. 8 and FIG. 9 respectively showed cell growth curve of JH016 and JH017 strain monitored with OD₅₉₅ with the changes of cell turbidities. The OD₅₉₅ values showed concentration-dependent decrease with the increase of alcohol concentration. Growth of JH016 was inhibited by 1-butanol at concentrations at least 2.5% (v/v), isobutanol at least 4%, 1-propanol at least 5%, isopropanol at least 5%, or ethanol at least 10%. And the growth of JH017 was inhibited by 1-butanol at concentrations at least 2% (v/v), isobutanol at least 3.5%, 1-propanol at least 5%, isopropanol at least 5%, or ethanol at least 12%. The abovementioned concentration of alcohol would for sure inhibit the growth of wild type E. coli.

### Example 7

### Morphology of E. Coli BW25113 and JH Strain by Transmission Electron Microscopy (TEM)

BW25113 and JH007 were cultured overnight and then inoculated into LB media with or without the addition of 30 μg/ml kanamycin and 50 μg/ml chloraphenicol. IPTG was added into the JH007 culture at the final concentration of 0.1 mM for 2 h. A drop of the cell culture was placed on a carbon-coated copper grid for 1 min and negative stained for 10 sec. The specimens were examined with an automatic transmission electron microscopes (Hitachi H-7650, Japan) operated at an accelerating voltage of 75 kV.

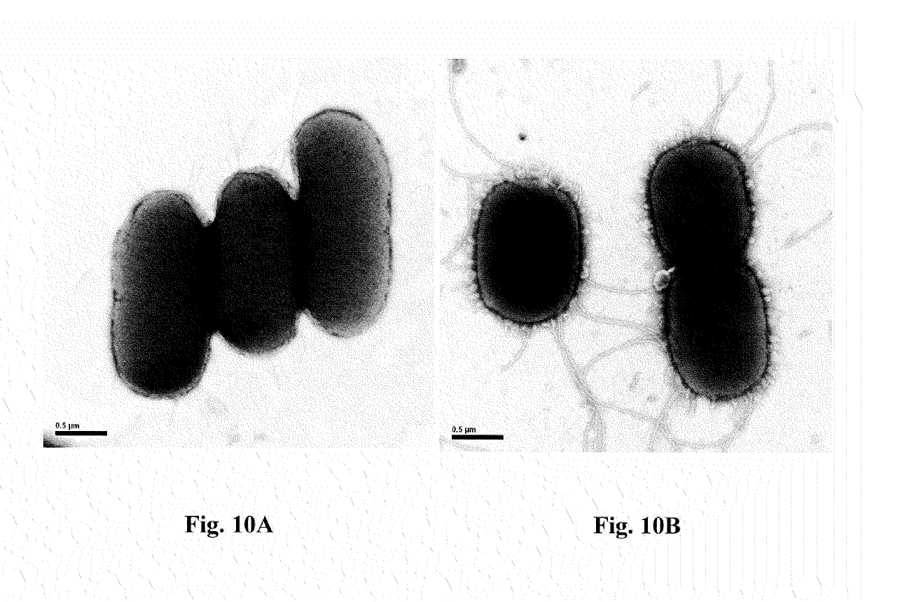

The morphology of wild type E. coli BW25113 and mutant E. coli JH007 was shown in FIG. 10a and FIG. 10b respectively. Significant alteration in JH007 was observed. E. coli JH007 had a rounder shape and a thicker cell wall than those of the wild type E. coli BW25113.

From the description and results of the abovementioned examples, the present invention successfully provided a novel alcohol-tolerant microorganism, which is able to tolerate butanol at a concentration as high as 5%. The morphology of this microorganism is significantly different from the wild type E. coli. These novel alcohol-tolerant microorganisms were obtained using synthetic systems biology. The abovementioned methods are examples for the present invention, which should not, however, be considered to limit the scope of the invention. Furthermore, the alcohol-tolerant E. coli in the present invention can tolerate isobutanol, 1-propanol, isopropanol, or ethanol as well, which will result in more industrial applications.

What is claimed is:

1. An alcohol tolerant E. coli selected from the group consisting of: BCRC 910400 (E. coli JH007), BCRC 910401 (E. coli JH016), and BCRC 910402 (E. coli JH017).

2. The alcohol tolerant E. coli as claimed in claim 1, wherein the E. coli JH007 is an ydhF⁻ mutant and overexpressing PhoH; the E. coli JH016 is a potG⁻ mutant and overexpressing hmp, and the E. coli JH017 is a potG⁻ mutant and overexpressing yqhD.

3. The alcohol tolerant E. coli as claimed in claim 1, wherein the alcohol is selected from the group consisting of: 1-butanol, isobutanol, 1-propanol, isopropanol, and ethanol.

4. The alcohol tolerant E. coli as claimed in claim 1, wherein the growth of the E. coli JH007 is inhibited by 1-butanol at least 5% (v/v).

5. The alcohol tolerant E. coli as claimed in claim 1, wherein the growth of the E. coli JH016 is inhibited by iso-butanol at least 4% (v/v).

6. A method for the preparation of the alcohol tolerant E. coli as claimed in claim 1 comprising:
(a) screening host cells having at least 20% higher survival rate than wild type in the presence of 2% 1-butanol treatment from E. coli single-gene deletion mutants, known as Keio Collection;
(b) transforming or transfecting a plasmid into the host cells, wherein the plasmid contains a gene of highly expressed 1-butanol tolerant protein; and
(c) isolating a transformant from transformed host cells with increased survival rate in the presence of 2% 1-butanol treatment.

7. The method as claimed in claim 6, wherein the host cell is an ydhF⁻ mutant.

8. The method as claimed in claim 6, wherein the host cell is a potG⁻ mutant.

9. The method as claimed in claim 6, wherein the plasmid is an IPTG-inducible pCA24N plasmid.

10. The method as claimed in claim 6, wherein the highly expressed 1-butanol tolerant protein is obtained from 2D electrophoresis analysis with 2% 1-butanol treated wild type followed by MS/MS analysis

11. The method as claimed in claim 6, wherein the gene of highly expressed 1-butanol tolerant protein is selected from the group consisting of PhoH, MdoG, YdfG, Hmp, YqhD, and TolB.

12. The method as claimed in claim 6, wherein the transformant has a rounder shape and a thicker cell wall than those of the wild type E. coli.

13. The method as claimed in claim 6, wherein the transformant is an ydhF⁻ mutant and overexpressing PhoH.

14. The method as claimed in claim 13, wherein the growth of the transformant is inhibited by 1-butanol at least 5% (v/v).

15. The method as claimed in claim 6, wherein the transformant is a potG⁻ mutant and overexpressing Hmp.

16. The method as claimed in claim 15, wherein the growth of the transformant is inhibited by iso-butanol at least 4% (v/v).

17. An method for enhancing 1-butanol tolerant of an E. coli comprising modifying the E. coli to increase expression of a gene selected from the group consisting of phoH, mdoG, ydfG, hmp, yqhD, and tolB.

18. The method as claimed in claim 17, wherein the E. coli is an ydhF⁻ mutant.

19. The method as claimed in claim 17, wherein the E. coli is a potG⁻ mutant.

20. The method as claimed in claim 17, wherein the 1-butanol concentration is from 2% to 5% (v/v).
